(19) United States
(12) Patent Application Publication
(10) Pub. No.: US 2003/0114342 A1
(43) Pub. Date: Jun. 19, 2003

(54) Compositions comprising quaternary ammonium compounds and dendritic polymers with antimicrobial activity

(75) Inventor: Larry Kent Hall, Nazareth, PA (US)

Correspondence Address: DARBY & DARBY P.C., Post Office Box 5257, New York, NY, 10150-5257, US

(73) Assignee: LONZA INC.

(21) Appl. No.: 10/245,814

(22) Filed: Sep. 17, 2002

Related U.S. Application Data
(60) Provisional application No. 60/324,174, filed on Sep. 20, 2001.

(51) Int. Cl. C11D 9/50

(52) U.S. Cl. 510/391; 510/475; 510/504

(57) ABSTRACT

The present invention provides compositions comprising quaternary ammonium compounds and dendritic polymers, which can be applied to hard surfaces. The compositions form transparent films and do not cause any unsightly residue on the surface to which is applied. The compositions provide immediate antimicrobial efficacy upon application as well as residual antimicrobial activity, especially broad-spectrum efficacy over prolonged periods, and may be used at low levels to prevent toxicity problems to the user. The film is water-soluble, easily removed completely with warm water, and does not require special techniques or harsh chemical treatments to effect its complete removal. The present invention also provides a method of disinfecting a substrate, preferably a hard surface, comprising applying a disinfecting composition of the invention to the substrate.

This application claims the benefit of U.S. Provisional Application No. 60/324,174, filed Sep. 20, 2001, which is hereby incorporated by reference.

### FIELD OF THE INVENTION

This invention relates to compositions comprising quaternary ammonium compounds and dendritic polymers, which form a transparent film on hard surfaces and provide both disinfectant efficacy upon application as well as sustained residual antimicrobial activity.

### BACKGROUND OF THE INVENTION

Pathogenic organisms, such as bacteria, fungi, and viruses, continue to cause infections in humans as well as domestic animals and pets. In recent years, there has been a particular growing concern over food-borne pathogens and the potential for them to contaminate the food chain. Disinfectant formulations have been developed over the last several decades to reduce or destroy pathogenic organisms and accordingly, reduce the rate of infection. Literally any hard surface including floors, walls, countertops, windows, windowsills, sinks, faucets, waste containers, appliances, and cabinet surfaces can become contaminated. Disinfectants have been developed to treat hard surfaces for use in hospitals, rest homes, schools, and homes.

Commonly used disinfectants include hypochlorite bleach and phenolic derivatives. While being very efficacious immediately upon application, these products provide little if any residual or substantive effects three to six hours after application. These products require re-application for continued disinfection.

Biocidal metallic materials, such as silver halide salts, have been utilized as disinfectants. U.S. Pat. No. 6,180,584 describes a water-insoluble polymeric film that provides sustained antimicrobial action containing a metallic salt and a polymer. The polymer composition described is very tightly bound to the hard surface that is treated and must be removed with dilute alcoholic base, making its removal problematic, especially in a household environment. U.S. Pat. No. 6,224,898 describes methods of treating wounds with a metal or metal-containing compound distributed on or in a dendritic polymer.

Quaternary ammonium compounds serve as the active antimicrobial agents in a wide variety of formulations, which are currently used in the household and in industrial and institutional markets. They are typically effective at low concentrations and provide a broad spectrum of bactericidal activity, against both gram positive and gram negative bacteria. The use of quaternary ammonium compounds as disinfectants is well known in the art, as for example, described in U.S. Pat. No. 5,421,898 and U.S. Pat. No. 6,080,387.

U.S. Pat. No. 5,421,898 describes a fabric substrate coated with the residue of an aqueous composition of water soluble polymer and a quaternary ammonium disinfectant, useful for controlling the release of the quaternary ammonium disinfectant. Water soluble polymers described are polyvinyl alcohol, polyvinylpyrrolidone, or poly(ethylene oxide). U.S. Pat. No. 6,080,387 describes the use of an aerosol antimicrobial composition formulated with an anionic polymer, a quaternary ammonium compound, and solvent. The composition, which is applied as an aerosol, provides disinfectancy immediately upon application as well as residual antimicrobial efficacy. The films produced, however, are generally not totally transparent and form a film residue, and therefore cannot be used on glass, polished metal, or other highly reflective surfaces.

With increased concerns over pathogens in the household environment, there has been a growing need for the development of novel formulations that can provide residual antimicrobial activity, especially broad-spectrum efficacy over prolonged periods without the necessity of hourly or daily re-application of the disinfectant, to increase efficacy, and to minimize maintenance costs. It is also desirable, especially in household applications, that the levels of antimicrobials employed be low and not cause any toxicity problems for the user.

### SUMMARY OF THE INVENTION

The present invention provides compositions comprising one or more quaternary ammonium compounds and dendritic polymers, which can be applied to hard surfaces. The compositions form transparent films and do not cause any unsightly residue on the surface to which it is applied. The compositions provide immediate antimicrobial efficacy upon application as well as residual antimicrobial activity, especially broad-spectrum efficacy over prolonged periods, and may be used at low levels to prevent toxicity problems to the user. The film is water-soluble, easily removed completely with warm water, and does not require special techniques or harsh chemical treatments to effect its complete removal.

The present invention also provides method of disinfecting a substrate, preferably a hard surface, comprising applying a disinfecting composition of the invention to the substrate.

### DETAILED DESCRIPTION OF THE INVENTION

The present invention relates to compositions comprising one or more quaternary ammonium compounds and dendritic polymers, which can be applied to hard surfaces. The film that is produced is efficacious against a broad range of microorganisms, including pathogenic organisms, and can provide efficacious antimicrobial performance for prolonged periods.

The quaternary ammonium compound may be any antimicrobial, antifungal, or antibacterial quaternary ammonium compound. Preferably, the quaternary ammonium compound effectively disinfects and/or sanitizes substrates. Suitable quaternary ammonium compounds include, but are not limited to, those having the formula:

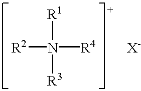

where R¹ is a linear or branched C₁-C₄ alkyl or linear or branched C₁-C₄ alkoxy; R² is a linear or branched C₁-C₁₈ alkyl or linear or branched C₁-C₁₈ alkoxy; R³ is a linear or branched C₆-C₁₈ alkyl or is —R⁵—O—R⁶—O(C₆H₅)R⁷, wherein R⁵ and R⁶ are independently linear or branched C₁-C₈ alkyl and R⁷ is linear or branched C₁-C ₁₂ alkyl; R⁴ is a linear or branched C₆-C₁₈ alkyl, benzyl, or (C₁-C₁₈ alkyl)benzyl; and X is an anion. According to a preferred embodiment, R³ and R⁴ are independently C₈-C₁₈ alkyl and more preferably C₈-C₁₂ alkyl and R₁ and R₂ are methyl. Suitable anions include, but are not limited to, halogens, such as chloride, bromide, and iodide, carbonates, hydroxides, saccharinates, phosphates, phosphonates, sulfates, bisulfates, alkylsulfates, and carboxylates.

Suitable quaternary ammonium compounds include, but are not limited to, alkyldimethylbenzyl ammonium chlorides, dialkylmethylbenzyl ammonium chlorides, dialkyldimethylammonium chlorides, alkyl dimethyl ethylbenzyl quaternary ammonium chlorides, benzethonium chloride (diisobutylphenoxyethoxyethyldimethylbenzyl ammonium chloride; available as Hyamine® 1622 from Lonza Inc. of Fair Lawn, N.J.), and any combination of any of the foregoing.

Non-limiting examples of alkyldimethylbenzyl ammonium chlorides include alkyl (C₁₄ 50%; C₁₂ 40%, C₁₆ 10%) dimethylbenzyl ammonium chloride (available as Barquat® MB-50 and MB-80 from Lonza Inc.), alkyl (C₁₄ 60%; C₁₆ 30%; C₁₂ 5%. C₁₈ 5%) dimethylbenzyl ammonium chloride (available as Barquat® 4280Z from Lonza, Inc.), (C₁₂-C₁₈alkyl) dimethylbenzyl ammonium chloride, and any combination of any of the foregoing.

Non-limiting examples of dialkyldimethyl ammonium chlorides include dioctyldimethylammonium chloride (available as Bardac® LF and LF-80 from Lonza, Inc.), octyldecyldimethylammonium chloride (available as a mixture of octyldecyldimethylammonium chloride, dioctyldimethylammonium chloride, and didecyldimethyl ammonium chloride as Bardac® 2050 and 2080 from Lonza, Inc.), didecyldimethylammonium chloride (available as Bardac® 2250 and 2280 from Lonza, Inc.), decylisononyldimethylammonium chloride (available as Bardac® 21 from Lonza, Inc.), diisodecyldimethylammonium chloride (available as BTC 99 from Stepan Co. of Northfield, Ill.), and any combination of any of the foregoing.

In a preferred embodiment, the dialkyldimethyl ammonium chloride is didecyldimethyl ammonium chloride.

The compositions of the invention comprise about 0.001 to about 99.0% by weight of one or more quaternary ammonium compounds, based upon 100% total weight of the composition. In a preferred embodiment, the compositions comprise about 0.01 to about 20.0% by weight of one or more quaternary ammonium compounds. In a more preferred embodiment, the compositions comprise about 0.01 to about 1.0% by weight of one or more quaternary ammonium compounds.

A variety of dendritic polymers including dendrimers may be used to form the compositions of the invention. Dendritic polymers are globular, branched structures, with molecule chains that branch out from a common center. A dendritic polymer includes several layers or generations of repeating units, which all contain one or more branch points. Each dendritic macromolecule includes a core cell, one or more layers of internal cells, and an outer layer of surface cells. The cells can be the same or different in chemical structure and branching functionality. The surface branched cells may contain either chemically reactive or passive functional groups. Chemically reactive surface groups can be used for further extension of dendritic growth or for modification of dendritic molecular surfaces. The chemically passive groups may be used to physically modify dendritic surfaces, such as to adjust the ratio of hydrophobic to hydrophilic terminals, and/or to improve the solubility of the dendritic polymer for a particular solvent. Chemical and physical characteristics of dendrimers, such as reactivity, complex or salt formation, hydrophilicity, can be varied and optimized. For instance, dendrimers may be derivatized with polar surface groups (carboxylate-, amino-, hydroxyl-, etc.) to make them soluble in polar solvents such as water, alcohol, and dimethylsulfoxide.

Dendritic polymers suitable for use with the invention also include macromolecules commonly referred to as cascade molecules, arborals and absorbent grafted molecules. Hyperbranched polymers represent a class of dendritic polymers, which contain high levels of non-ideal irregular branching as compared with the more nearly perfect regular structure of dendrons or dendrimers. Specifically, hyperbranched polymers contain a relatively high number of irregular branching areas in which not every repeat unit contains a branch juncture.

Suitable dendritic polymers include bridged dendritic polymers, wherein dendritic macromolecules are linked together either through surface functional groups or through a linking molecule. The individual dendritic polymers can be aggregated together through physical or non-covalent interactions or covalent bonding to form larger clusters if desired.

Dendritic polymers include poly(amidoamine) (PAMAM) dendrimers, polypropylene amine (POMAM) dendrimers, poly(amido)alcohols, polyether, polyamine, and polyester polymers.

Preferred dendritic polymers are hyperbranched polyesteramides (hybrane polymers), which are synthesized by polycondensation of a monomer prepared by the addition reaction of diisopropanolamine and an anhydride. Anhydrides that are preferred are hexahydrophthalic anhydride, succinic anhydride, alkenylsuccinic anhydride, and phthalic anhydride. Hybrane polymers may be derivatized with hydroxy, aliphatic, and aromatic carboxylic ester, carboxylic acid, unsaturated fatty acid esters, polyethylene oxide, and tertiary amine end groups. In a more preferred embodiment, the hyperbranched polyesteramides are terminated with hydroxyls or the amine, —N(CH₃)₂. The average molecule weight of hybrane polymers is 1000-5000, with a broad molecular weight distribution. The polymers are thermally stable up to 250° C. and stable to hydrolysis at pH 4-10.

Dendrimers can be prepared using divergent or convergent synthesis using methods well known in the art. In the divergent method, one starts at a central multifunctional core and step by step layers (generations) are built around the core. In each generation, the number of functional groups in the outermost layer increases exponentially with the generation number. The second approach is the convergent approach, in which dendrimer segments are built up from the outside towards the focal unit.

Examples of dendrimers, dendrons, hyperbranched polymers, and methods of synthesizing the same are set forth in U.S. Pat. Nos. 4,507,466, 4,558,120, 4,568,737, 4,587,329, 4,631,337, 4,694,064, 4,713,975, 4,737,550, 4,871,779, 4,857,599, 5,041,516, 5,530,092, and 5,418,301, and International Publication Nos. WO 95/20619, WO 98/52995, and WO 95/02008, all of which are incorporated herein in their entirety.

The compositions of the invention comprise from about 1.0 to about 99.999% by weight of dendritic polymer, based upon 100% total weight of composition. In a preferred embodiment, the compositions comprise about 80.0% to about 99.99% dendritic polymer. In a more preferred embodiment, the compositions comprise about 99.0% to about 99.99% dendritic polymer.

In one embodiment, the composition of the invention further comprises a solvent. In one embodiment, the solvent may be water. In another embodiment, the solvent may be mixtures of ethanol, propylene glycol, isopropanol, or other alcohols and water. In one embodiment, ethanol is present in 48-50% by weight, based on 100% total weight of composition.

The compositions of the invention can also include additives, such as chelators, builder salts, dyes, fragrances, nonionic surfactants, wetting agents, and perfluorosurfactants, such as those commonly used in the art of cleaning and disinfecting solutions. The compositions of the invention can also include additives such as a leveling agent, such as those commonly used in the art of coatings or paints.

Non-limiting examples of chelators include citric acid, nitriloacetic acid, phosphoric acids, zeolites, EDTA (ethylene diamine tetra acetic acid), and any combination of any of the foregoing. The compositions generally contains from about 0.1 to about 10% and preferably from about 1.0 to about 7.0% by weight of chelator, based upon 100% total weight of composition.

Non-limiting examples of builder salts include sodium metasilicate, sodium tripolyphosphate, sodium nitrilotriacetate, sodium carbonate, sodium silicate, citric acid salts, zeolites, and any combination of any of the foregoing. A composition generally contains from about 0.1 to about 15% and preferably from about 0.5 to about 2.0% by weight of builder salt, based upon 100% total weight of composition.

Suitable nonionic surfactants include, but are not limited to, amine oxides, nonylphenol ethoxylates, linear alcohol ethoxylates, secondary alcohol ethoxylates, ethoxylated propoxylated (EOPO) block polymers, and any combination of any of the foregoing. The compositions generally comprise from about 0.1 to about 25% and preferably from about 0.5 to about 10% by weight of nonionic surfactant, based upon 100% total weight of composition. Perfluorosurfactants may be included in the compositions as an additive. The compositions generally contain from about 0.001% to about 0.25% perfluorosurfactants.

Leveling agents include, but are not limited to, amine oxides and hydrocarbon and fluorocarbon surfactants. The compositions generally contain from about 0.1 to about 2.5% leveling agent.

The compositions of the invention may be prepared by any method known in the art. For example, they may be prepared by mixing one or more quaternary ammonium compounds with a solvent and then adding the dendritic polymer.

The invention is directed to a method of disinfecting a substrate comprising applying a disinfecting composition comprising a composition of the invention, to the substrate. In a preferred embodiment, the substrate is a hard surface. In a further embodiment, the hard surface is any hard surface found in the home or an industrial or institutional setting. In another embodiment, the hard surface is a floor, wall, countertop, appliance, or fixture.

Substrates, which may be disinfected with the compositions, include, but are not limited to, those located in dairies, homes, health care facilities, swimming pools, canneries, food processing plants, restaurants, hospitals, institutions, and industry, including secondary oil recovery. Hard surfaces, such as glass and polished aluminum, are particularly suited for application. Specific areas targeted for application include hard surfaces in the home such as kitchen countertops, cabinets, appliances, waste cans, laundry areas, garbage pails, bathroom fixtures, toilets, water tanks, faucets, mirrors, vanities, tubs, and showers. The compositions can also be used to sanitize floors, walls, furniture, mirrors, toilet fixtures, windows, and wood surfaces, such as fence rails, porch rails, decks, roofing, siding, window frames, and door frames. The compositions are particularly well suited for application on indirect food contact surfaces, such as cutting boards, utensils, containers, dishes, wash basins, appliances, and countertops. The compositions can be used to sanitize diary plant equipment, milking machines, milk pails, tank trucks, and the like. Areas in hospitals would include beds, gurneys, tables, canisters, toilets, waste cans, stands, cabinets, shower stalls, floors, walls or any other non-porous surface.

The compositions may be applied to a substrate in order to disinfect the substrate. The composition may be applied by any method known in the art including, but not limited to, brushing, spraying, soaking, aerosolizing, mopping, wiping, and the like. For example, the composition may be absorbed or incorporated into wipes, which are then rubbed against a substrate. In another example, the film can be applied as a spray or as an aerosol. The composition may also be used in clean-in-place applications, i.e., by recirculating the compositions in pipes, kettles, tanks, and the like to disinfect the same.

Compositions may be in the form of a concentrate, wherein the active concentrate is diluted with a solvent, such as water, propylene glycol, or ethanol or mixtures thereof, before use. For example, the concentrate composition may be diluted 1:100 of its original concentration. The final concentration of quaternary ammonium compound is usually 150 ppm to 1000 ppm.

The following examples are illustrative of the present invention, however, it will be understood that the invention is not limited to the specific details set forth in the examples. All percentages are by weight unless otherwise indicated.

### EXAMPLE 1

The test compositions in Table 1 were prepared by mixing the quaternary ammonium compound, didecyldimethyl ammonium chloride (DDAC), with water in the concentrations indicated. Dendritic polymer, Hybrane® (P/S 80 (a hyperbranched poly-esteramide available from DSM N.V.), was added in the concentrations indicated. The mixture was stirred magnetically for 10 minutes and transferred to a glass bottle. The test compositions (35 μL) were applied to a glass slide and allowed to air-dry overnight in a large, covered petri-dish. No test composition was applied to the control slide. 10 μL of inoculum (P. aeruginosa) was evenly applied to each slide. The slides were placed in a large covered petri-dish and incubated in a humidified atmosphere at room temperature for one hour. Slides were then scraped to lift any residual cells, diluted 1:10 three times in Letheen Broth, and inoculated onto plates. The plates were incubated for 24 hours, colonies were counted, and then the colonies were compared to the control to determine the efficacy of the film. The control slide had an average cfu of 1,500,000. The results are shown in Table 1.

TABLE 1

| | | | % reduction in |
|---|---|---|---|
| | | Hybrane ® | organisms (compared |
| | DDAC (ppm) | P/S 80 (ppm) | to control) |
| Experiment 1 | 10,000 | 20,000 | 99.0 |
| Experiment 2 | 1,000 | 10,000 | 99.8 |
| Experiment 3 | 100 | 1,000 | 99.9 |

A significant reduction in the microbial count compared to the control was observed when the quaternary ammonium compound/dendritic polymer film was used.

### EXAMPLE 2

The test composition in Table 2 was prepared by mixing the quaternary ammonium compound, didecyldimethyl ammonium chloride (DDAC), with water in the concentrations indicated. Dendritic polymer, Hybrane® HA-1690 (a hyperbranched poly-esteramide available from DSM N.V.), was added in the concentrations indicated. The mixture was stirred magnetically for 10 minutes and transferred to a glass bottle. The test composition (35 μL) was applied to a glass slide and allowed to air-dry overnight in a large, covered petri-dish. No test composition was applied to the control slide. 10 μL of inoculum (P. aeruginosa) was evenly applied to the slide. The slide was placed in a large covered petri-dish and incubated in a humidified atmosphere at room temperature for one hour. The slide was then scraped to lift any residual cells, diluted 1:10 three times in Letheen Broth, and inoculated onto a plate. The plate was incubated for 24 hours, colonies were counted, and then the colonies were compared to the control to determine the efficacy of the film. The control slide had an average cfu of 1,500,000. The results are shown in Table 2.

TABLE 2

| | | Hybrane ® | % reduction in |
|---|---|---|---|
| | | HA-1690 | organisms (compared |
| | DDAC (ppm) | (ppm) | to control) |
| Experiment 4 | 100 | 1,000 | 99.9 |

A significant reduction in the microbial count compared to the control was observed when the quaternary ammonium compound/dendritic polymer film was used.

### EXAMPLE 3

The test compositions in Table 3 were prepared by mixing the quaternary ammonium compound, didecyldimethyl ammonium chloride (DDAC), with water in the concentrations indicated. Dendritic polymer, Hybrane® P/S 80 or Hybrane® HA-1690 (hyperbranched poly-esteramides available from DSM N.V.), was added in the concentrations indicated.

Ethanol was added in the concentration by weight indicated, to reduce viscosity. The mixture was stirred magnetically for 10 minutes and transferred to a glass bottle. The test compositions (35 μL) were applied to a glass slide and allowed to air-dry overnight in a large, covered petri-dish. No test composition was applied to the control slide. 10 μL of inoculum (P. aeruginosa) was evenly applied to each slide. The slides were placed in a large covered petri-dish and incubated in a humidified atmosphere at room temperature for one hour. Slides were then scraped to lift any residual cells, diluted 1:10 three times in Letheen Broth, and inoculated onto plates. The plates were incubated for 24 hours, colonies were counted, and then the colonies were compared to the control to determine the efficacy of the film. The control slide had an average cfu of 1,500,000. The results are shown in Table 3.

TABLE 3

| | | | | | % reduction in |
|---|---|---|---|---|---|
| | | Hybrane ® | Hybrane ® | Ethanol | organisms |
| | DDAC | P/S 80 | HA-1690 | (% | (compared to |
| | (ppm) | (ppm) | (ppm) | weight) | control) |
| Experi- | 10,000 | 20,000 | 0 | 48.5 | 99.0 |
| ment 5 | | | | | |
| Experi- | 1,000 | 0 | 1,000 | 49.9 | 99.8 |
| ment 6 | | | | | |
| Experi- | 500 | 2,000 | 0 | 49.9 | 97.8 |
| ment 7 | | | | | |

A substantial reduction in the microbial count compared to the control was observed when the quaternary ammonium compound/dendritic polymer film was used.

### EXAMPLE 4

The test compositions in Table 4 were prepared by mixing the quaternary ammonium compound, didecyldimethyl ammonium chloride (DDAC), with water in the concentrations indicated. Dendritic polymer, Hybrane® P/S 80 or Hybrane(t HA-1690 (hyperbranched poly-esteramides available from DSM N.V.), was added in the concentrations indicated. Ethanol was added in the concentration by weight indicated, to reduce viscosity. The mixture was stirred magnetically for 10 minutes and transferred to a glass bottle. Films were prepared on glass slides by placing a small drop of test substance from a transfer pipette directly onto the slide. The control is a commercial disinfectant aerosol spray marketed as “Clorox Disinfectant Spray” (available from Clorox Company; contains 0.63% quaternary ammonium compounds, 65.0% ethanol, and 34.37% inert ingredients). The control aerosol was sprayed into a beaker and dropped onto the glass slide using a transfer pipette in the same way as the test substances. The appearance of the films produced were compared. The results are shown in Table 4.

TABLE 4

| | | | | | Quaternary | |
|---|---|---|---|---|---|---|
| | | | | | ammonium | |
| | | | | | content of | Film |
| | | Hybrane ® | Hybrane ® | Ethanol | Clorox | appearance |
| | DDAC | P/S 80 | HA-1690 | (% | disinfectant | on glass |
| | (ppm) | (ppm) | (ppm) | weight) | spray (ppm) | surface |
| Exp. 8 | 10,000 | 20,000 | 0 | 48.5 | 0 | Clear; very |
| | | | | | | fine |
| | | | | | | streaking; |
| | | | | | | dry |
| Exp. 9 | 1,000 | 0 | 1,000 | 49.9 | 0 | Clear; dry |
| Exp. 10 | 500 | 2,000 | 0 | 49.9 | 0 | Clear; dry |
| Exp. 11 | 0 | 0 | 0 | 65.0 | 6300 | Opaque; |
| (Control) | | | | | | tacky to |
| | | | | | | touch |

The quaternary ammonium compound/dendritic polymer films formed were transparent as compared to the opaque film formed using the control commercial disinfectant spray.

All patents, applications, articles, and publications mentioned above are hereby incorporated by reference.

Many variations of the present invention will suggest themselves to those skilled in the art in light of the above detailed description. Such obvious variations are within the full intended scope of the appended claims.

1. A composition comprising:
a) one or more quaternary ammonium compounds, and
b) a dendritic polymer.

2. The composition of claim 1, wherein the composition comprises from about 0.001 to about 99.0% by weight of the quaternary ammonium compounds, based upon 100% total weight of composition.

3. The composition of claim 2, wherein the composition comprises about 0.01 to about 20.0% by weight of the quaternary ammonium compounds.

4. The composition of claim 3, wherein the compositions comprises about 0.01 to about 1.0% by weight of the quaternary ammonium compounds.

5. The composition of claim 1, wherein one of the quaternary ammonium compounds has the formula: 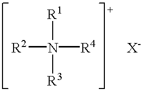
wherein
R¹ is a linear or branched C₁-C₄ alkyl or linear or branched C₁-C₄ alkoxy;
R² is a linear or branched C₁-C₁₈ alkyl or linear or branched C₁-C₁₈ alkoxy;
R³ is a linear or branched C₆-C₁₈ alkyl or is —R⁵—O—R⁶—O(C₆H₅)R⁷;
R⁴ is a linear or branched C₆-C₁₈ alkyl, benzyl, or (C₂-C₁₈ alkyl)benzyl;
R⁵ and R⁶ are independently linear or branched C₁-C₈ alkyl;
R⁷ is linear or branched C₁-C₁₂ alkyl; and
X is an anion.

6. The composition of claim 5, wherein the quaternary ammonium compound is selected from the group consisting of alkyldimethylbenzyl ammonium chloride, dialkylmethylbenzyl ammonium chloride, dialkyldimethylammonium chloride, alkyldimethylethylbenzyl ammonium chloride, diisobutylphenoxyethoxyethyl chloride, and any combination of any of the foregoing.

7. The composition of claim 6, wherein the dialkyldimethyl ammonium chloride is selected from the group consisting of dioctyldimethylammonium chloride, octyldecyldimethylammonium chloride, didecyldimethyl ammonium chloride, decylisononyldimethylammonium chloride, diisodecyldimethylammonium chloride, and any combination of any of the foregoing.

8. The composition of claim 7, wherein the dialkyldimethyl ammonium chloride is didecyldimethyl ammonium chloride.

9. The composition of claim 1, wherein the composition comprises from about 1.0 to about 99.999% by weight of the dendritic polymer, based upon 100% total weight of composition.

10. The composition of claim 9, wherein the composition comprises about 80.0% to about 99.99% dendritic polymer.

11. The composition of claim 10, wherein the composition comprises from about 99.0% to about 99.99% dendritic polymer.

12. The composition of claim 1, wherein the dendritic polymer is a hyperbranched polymer.

13. The composition of 12, wherein the hyperbranched polymer is a hyperbranched polyesteramide polymer.

14. The composition of claim 13, wherein the polyesteramide polymer has either hydroxy or —N(CH₃)₂ end groups.

15. The composition of claim 1, wherein the dendritic polymer is a poly(amidoamine) dendrimer, a polypropylene amine dendrimer, a poly(amido) alcohol dendrimer, a polyether dendrimer, a polyamine dendrimer, or a polyester dendrimer.

16. The composition of claim 1, which further comprises a solvent.

17. The composition of claim 16, wherein the solvent is selected from the group consisting of water, ethanol, isopropanol, and propylene glycol.

18. The composition of claim 1, wherein the composition further comprises at least one of a chelator, a builder salt, a dye, a fragrance, a nonionic surfactant, a perfluorosurfactant, and a leveling agent.

19. A method of disinfecting a substrate comprising applying a disinfecting composition comprising the composition of claim 1 to the substrate.

20. The method of claim 19, wherein the substrate is a hard surface.

21. The method of claim 20, wherein the hard surface is any hard surface found in the home or an industrial or institutional setting.

22. The method of claim 21, wherein the hard surface is a floor, wall, countertop, appliance, or fixture.

23. The method of claim 20, wherein the hard surface is a surface found in a hospital.

24. The method of claim 23, wherein the hard surface is a bed, table, canister, toilet, waste can, stand, cabinet, shower stall, floor, or wall.

25. The method of claim 23, wherein the hard surface is a countertop, appliance, wall, floor, toilet, water tank, faucet, mirror, or window.

26. The method of claim 19, wherein the disinfecting solution is applied by brushing, spraying, soaking, aerosolizing, mopping, or wiping.
